(19) United States
(12) Patent Application Publication
(10) Pub. No.: US 2005/0070612 A1
(43) Pub. Date: Mar. 31, 2005

(54) Methods and compositions for inhibiting the proliferation of prostate cancer cells

(75) Inventor: Charles Y.F. Young, Rochester, MN (US)

Correspondence Address: FISH & RICHARDSON P.C., 3300 DAIN RAUSCHER PLAZA, 60 SOUTH SIXTH STREET, MINNEAPOLIS, MN, 55402, US

(73) Assignee: Mayo Foundation for Medical Education and Research , a Minnesota corporation

(21) Appl. No.: 10/915,662

(22) Filed: Aug. 10, 2004

Related U.S. Application Data
(63) Continuation of application No. 09/957,006, filed on Sep. 20, 2001, now abandoned.

(51) Int. Cl. A61K 31/045

(52) U.S. Cl. 514/724

(57) ABSTRACT

The invention provides for methods of monitoring the proliferation of cultured prostate cancer cells in the presence of POH, methods of treating an individual with prostate cancer or at risk of developing prostate cancer, and methods of reducing the risk of recurrence of prostate cancer in an individual who had previously been treated for prostate cancer. Methods of the invention further include treating an individual with benign prostatic hyperplasia (BPH) with POH as well as methods of screening for compounds that inhibit the proliferation of prostate cancer cells. The invention provides for compositions and articles of manufacture containing POH in particular formulations, and POH with a second compound that also exerts an effect on the androgen receptor.

### FEDERALLY SPONSORED RESEARCH OR DEVELOPMENT

The U.S. Government may have certain rights in this invention pursuant to NIH grant DK41995 and Army Defense grant DAMD17-98-1-8523.

### TECHNICAL FIELD

This invention relates to prostate cancer, and more particularly to methods and compositions for inhibiting the proliferation of prostate cancer cells.

### BACKGROUND

The prostate gland is located between the bladder and the rectum and wraps around the urethra. The prostate is composed of glandular tissue that produces a milky fluid and smooth muscles that contract during sex and squeeze this fluid into the urethra where it mixes with other fluid and sperm to form semen. The prostate gland converts testosterone to a more powerful male hormone, dihydrotestosterone, which affects the size of the gland and plays an important role in prostate cancer.

Prostate cancer is a malignant tumor that arises in the prostate gland and can eventually spread through the blood and lymph fluid to other organs, bones, and tissues. Prostate cancer is the most commonly diagnosed cancer in the U.S., and it is the second leading cause of cancer death in American men after non-melanoma skin cancer. Although prostate cancer is just as common in Japan as in the United States, death rates from prostate cancer are significantly lower in Japan. It is unlikely that these differences are all genetic, because Japanese men who migrate to the United States die of prostate cancer with increasing frequency as a function of the number of years they reside in the United States. It is possible that this paradox could be explained, at least in part, by dietary factors.

Benign prostatic hyperplasia (BPH) is a benign enlargement of the prostate gland caused by the growth of both glandular and stromal tissues. Because the prostate enlargement in BPH is affected by testosterone, many men are concerned that it may be related to prostate cancer. A ten-year study, however, found no higher risk for prostate cancer in men with or that have experienced BPH. BPH develops in the inner zone of the prostate (i.e., predominantly stromal cells), while cancer tends to develop in the outer area (i.e., epidermal cells).

### SUMMARY

It is reported herein that the transactivating ability of the androgen receptor was inhibited by POH. Accordingly, the invention provides for methods of monitoring the proliferation of cultured prostate cancer cells in the presence of POH, methods of treating an individual with prostate cancer or at risk of developing prostate cancer, and methods of reducing the risk of recurrence of prostate cancer in an individual who had previously been treated for prostate cancer. The invention further includes methods of treating an individual with benign prostatic hyperplasia (BPH) as well as methods of screening for compounds that inhibit the proliferation of prostate cancer cells. The invention provides for compositions and articles of manufacture containing POH in particular formulations, or POH with a second compound that also exerts an effect on the androgen receptor.

In one aspect, the invention provides methods of monitoring the proliferation of cultured prostate cancer cells in the presence of perillyl alcohol (POH). Such a method includes contacting the prostate cancer cells with POH or a derivative thereof and determining the transactivating ability of an androgen receptor. Generally, a decrease in the transactivating ability of the androgen receptor indicates an inhibitory effect by POH on the proliferation of the prostate cancer cells. Representative prostate cancer cell lines include LNCaP cells or LAPC-4 cells.

In another aspect, the invention provides methods of treating an individual with prostate cancer or at risk of developing prostate cancer. Methods of treating an individual with prostate cancer or at risk of developing prostate cancer include identifying an individual with prostate cancer or at risk of developing prostate cancer, administering a dose of perillyl alcohol (POH) or a derivative thereof to the individual that is effective to inhibit the transactivating ability of an androgen receptor, and monitoring the transactivating ability of the androgen receptor in the individual. Inhibiting the transactivating ability of the androgen receptor inhibits the proliferation of prostate cancer cells, thereby treating the individual. For example, POH can be administered to a human, and in an amount of from about 100 mg/kg to about 300 mg/kg. POH can be administered orally, transdermally, intravenously, intraperitoneally, or using an implant.

In still another aspect, the invention provides for methods of reducing the risk of recurrence of prostate cancer in an individual who previously had been treated for prostate cancer. Such a method includes the step of administering a dose of perillyl alcohol (POH) or a derivative thereof to the individual that is effective to inhibit the transactivating ability of an androgen receptor. The method can further include the step of monitoring the transactivating ability of the androgen receptor in the individual. Generally, inhibiting the transactivating ability of the androgen receptor inhibits the proliferation of prostate cancer cells, and thereby reduces the risk of recurrence of prostate cancer in the individual. The individual may have previously undergone a radical prostectomy.

In yet another aspect, the invention provides methods of treating an individual with benign prostatic hyperplasia (BPH). This method includes identifying an individual with BPH, and administering a dose of perillyl alcohol (POH) or a derivative thereof to the individual that is effective to inhibit the transactivating ability of an androgen receptor. The method also can include monitoring the transactivating ability of the androgen receptor in the individual. Inhibiting the transactivating ability of the androgen receptor thereby treats the BPH in the individual.

The invention additionally provides methods of screening for compounds that inhibit the proliferation of prostate cancer cells, including contacting prostate cancer cells with a compound, and determining the transactivating ability of an androgen receptor. The method also can include monitoring the transactivating ability of the androgen receptor in the prostate cancer cells. Decreased transactivating ability of the androgen receptor in the prostate cancer cells compared to prostate cancer cells not contacted with the compound indicates a compound that inhibits the proliferation of prostate cancer cells. Prostate cancer cells such as LNCaP cells orLAPC-4 cells can be used in this method.

Further, the invention provides compositions that include perillyl alcohol (POH) or a derivative thereof, one or more compounds that has a particular mechanism of action (i.e., inhibiting expression of a gene encoding an androgen receptor, inhibiting nuclear localization of an androgen receptor, and inhibiting the transactivating ability of an androgen receptor) and a pharmaceutically acceptable carrier. Representative examples of compounds having such particular mechanisms of action include silymarin, silibin, docosahexaenoic acid (DHA), eicosapentaenoic acid (EPA), quercetin, resveratrol, flufenamic acid, tea polyphenols, and anti-androgen compounds. It is a feature of the invention to provide such a composition in the form of an article of manufacture (e.g., a kit). Such an article of manufacture can include packaging material comprises instructions for using the composition to inhibit the transactivating ability of an androgen receptor in an individual.

In another aspect of the invention, there are provided compositions that include perillyl alcohol (POH) or a derivative thereof and that are formulated for transdermal delivery to the prostate of an individual. Delivery to the prostate typically inhibits the transactivating ability of an androgen receptor. In addition, the invention provides compositions that include perillyl alcohol (POH) or a derivative thereof and that are formulated for implantation near the prostate of an individual. Generally, implantation near the prostate inhibits the transactivating ability of an androgen receptor.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 depicts the effects of POH on androgen-stimulated proliferation responses in LNCaP cells. *Significant inhibition compared to the no treatment control.

FIG. 2 depicts the androgen-induced expression of PSA and hK2 protein in LNCaP cells in the presence of POH. *Significant inhibition compared to the no treatment controls.

FIG. 3 depicts the effects of POH on the androgen receptor-mediated transcription of a heterologous reporter gene. *Significant inhibition compared to the no treatment controls.

FIG. 4 depicts the inhibition of the expression of the androgen receptor gene at the transcriptional level by POH.

FIG. 5 depicts the inhibition of the expression of the androgen receptor gene at the translational level by POH.

FIG. 6 depicts the effects of POH treatment on the expression of the c-jun protein.

### DETAILED DESCRIPTION

It is reported herein that the transactivating activity of the androgen receptor was inhibited by POH. Accordingly, the invention provides for methods of monitoring the proliferation of cultured prostate cancer cells in the presence of POH, methods of treating an individual with prostate cancer or at risk of developing prostate cancer, and methods of reducing the risk of recurrence of prostate cancer in an individual who had previously been treated for prostate cancer. The invention further includes methods treating an individual with benign prostatic hyperplasia (BPH) as well as methods of screening for compounds that inhibit the proliferation of prostate cancer cells. The invention provides for compositions and articles of manufacture containing POH in particular formulations, or POH with a second compound that also exerts an effect on the androgen receptor.

It was shown herein that POH inhibited androgen-stimulated secretion of both prostate-specific antigen (PSA) and hK2. The transactivating ability of the androgen receptor was diminished by POH. The invention provides a novel aspect of POH in that POH can attenuate androgen receptor-mediated transactivation of prostate cancer-specific genes in androgen-responsive prostate cancer cells. Thus, the invention provides for methods of preventing or treating prostate cancer using POH.

The Androgen Receptor and Prostate Cancer

Androgens play an important role in the proliferation, differentiation, maintenance, and function of the prostate. The androgen receptor is the essential mediator for androgen action and is a ligand-dependent transcription factor belonging to the nuclear steroid hormone receptor superfamily. Androgens can enhance androgen receptor protein levels by increasing the half-life, as well as by stimulating the phosphorylation of the androgen receptor. Phosphorylation may affect numerous characteristics of nuclear receptors including ligand binding, nuclear translocation, dimerization, DNA binding, and protein-protein interactions.

Evidence shows that androgens are also involved in the development and progression of prostate cancer. Therefore, the androgen receptor also plays a critical role in the development of prostate cancer, in part due to overstimulation of the receptor by androgens. Prostate cancer also has been attributed to altered transactivation activities of the receptor or to mutations in the androgen receptor that, for example, enable the receptor to respond to non-androgen steroids. The androgen receptor can be expressed in all stages of prostate cancer, and at least-one-third of advanced prostate cancers contain amplified androgen receptor genes.

The utilization of androgen deprivation as a treatment for advanced prostate cancer was first demonstrated in 1941 and has become a standard treatment. Based on the morbidity associated with ablation of the adrenal glands, castration alone was the gold standard until the 1980s, when anti-androgen agents, including cyproterone acetate, megestrol acetate, and flutamide, were developed to compete with androgen for binding to the androgen receptor. Many new classes of drugs that interfere with androgen production and function have been identified.

In spite of the apparent regression of tumors by hormone therapy, however, prostate cancer often recurs within 3 years and becomes hormone refractory with a potentially fatal outcome. Many molecular mechanisms have been postulated to be responsible for the development of recurrent hormone-refractory tumors with most involving alterations in the function of the androgen receptor and its complex signaling pathways. The androgen receptor can be activated by a number of growth factors or cytokines in the absence of androgens or by low levels of androgens or other non-androgenic steroid hormones after hormone therapy. That the majority of hormone-refractory cancers still express the androgen-responsive prostate-specific antigen PSA is a protein secreted by the epithelial cells of the prostate gland, including prostate cancer cells. An abnormally high level of PSA is indicative of abnormal prostate cells. (PSA) gene indicates that the androgen receptor signaling pathway is functional.

Nucleic acid sequences encoding androgen receptors have been cloned and sequenced from numerous organisms. Representative organisms and GenBank accession numbers for androgen receptor sequences therefrom include the following: frog (Xenopus laevis, U67129), mouse (Mus musculus, 109558), rat (Rattus norvegicus, 292896), human (Homo sapiens, 105325), rabbit (Oryctolagus cuniculus 577829), cow (Bos taurus, Z75313, Z75314, Z75315), canary (Serinus canaria, 414734), and whiptail lizard (Cnemidophous uniparens, 1195596). Additionally, Cancer Genetics Web (www.cancer-genetics.org) contains database entries for wild-type and mutant androgen receptor sequences.

Perillyl Alcohol

Perillyl alcohol (POH) is the hydroxylated form of D-limonene. Both are monocyclic monoterpenes. Monoterpenes are found in essential oils of many plants including lemons, oranges, grapefruit, caraway, dill, bergamot, peppermint, spearmint, grasses and tomatoes. Monoterpenes are also associated with vegetables and some evergreen trees. POH is often distilled from lavender, is found in citrus fruits cherries, mint, celery seeds, and can be produced synthetically. It is typically used as a flavoring agent, food additive, and fragrance and has been found to be a major volatile component of mother's milk.

POH can inhibit cell cycle progression, the activity of small G protein, and the post-translational isoprenylation of Ras. POH can induce the expression of glutathione S-transferase, insulin-like growth factor-2 receptor, transforming growth factor beta-1/receptor and AP-1. POH also induces apoptosis of cells in a rat mammary tumor model. POH has been used in human phase I clinical trials for advanced malignancies, and the primary metabolites found were perillic acid and dihydroperillic acid. Therefore, derivatives of POH, including perillic acid and dihyroperillic acid, are useful in the invention.

Methods of Monitoring and Inhibiting the Proliferation of Prostate Cancer Cells

The invention provides for methods of monitoring the proliferation of prostate cancer cells. According to the methods of the invention, the proliferation of prostate cancer cells can be monitored by contacting those cells with POH and then determining the transactivating ability of the androgen receptor using conventional methods (e.g., methods described herein). A decrease in the transactivating ability is indicative of an inhibitory effect by POH on the proliferation of the prostate cancer cells. Proliferation of prostate cancer cells as used herein refers to an increase in the number of prostate cancer cells (in vitro or in vivo) over a given period of time (e.g., hours, days, weeks, or months). It is noted that the number of prostate cancer cells is not static and reflects both the number of cells undergoing cell division and the number of cells dying (e.g., by apoptosis). An inhibition of the proliferation of prostate cancer cells can be defined as a decrease in the rate of increase in prostate cancer cell number, a complete loss of prostate cancer cells, or any variation therebetween. With respect to tumors, a decrease in the size of a tumor can be an indication of an inhibition of proliferation.

Prostate cancer cells that can be maintained in culture and are useful in the invention include without limitation LNCaP cells and LAPC-4 cells. The LNCaP cell line is an established androgen-responsive prostate cancer cell line obtained from a lymph node metastasis of a prostate cancer patient. LNCaP cells express the androgen receptor and a number of androgen-inducible genes such as PSA, human glandular kallikrein (hK2), NKX3.1 and ornithine decarboxylase (ODC). The gene encoding the androgen receptor in the LNCaP cell line contains a mutation in its ligand-binding domain, but otherwise is functional. LAPC-4 cells, another androgen responsive prostate cancer cell line suitable for use in the invention, expresses a wild-type androgen receptor. LAPC-4 cells additionally express PSA and hK2, which are up-regulated in the LAPC-4 cells by androgens. Other prostate cancer cell lines are available and include PC-3 and DU145.

The invention further provides for methods of treating an individual with prostate cancer or at risk of developing prostate cancer. An individual is first identified as having prostate cancer or being at risk for developing prostate cancer and then administered an effective dose of POH. The transactivating ability of the androgen receptor can be monitored in the individual to evaluate the effects of POH on prostate cancer cells. Generally, an inhibition of the transactivating ability of the androgen receptor by POH inhibits the proliferation of prostate cancer cells, thereby treating the individual.

Prostate cancer cells can be identified using several criteria. Prostate cancer cells in culture (e.g., LNCaP cells) can be characterized by the response of such cells to androgens or to androgenic agonists or antagonists. Molecular markers, such as increased or decreased expression of androgen-regulated genes or genes involved in prostate cancer (e.g., PSA, hk2, c-jun, ODC, and NKX3.1) also can be used to characterize prostate cancer cells in culture. Prostate cancer in vivo can be identified by a digital rectal examination of a patient, or by imaging or scanning techniques (e.g., magnetic resonance imaging (MRI), or prostascint scans). In addition, the degree of cellular differentiation can be evaluated in prostate cancer cells from an individual, typically removed via a biopsy of prostate tissue, using a Gleason score. Further, there are several commercially available diagnostic tests for PSA and PSA-II (e.g., Roche Diagnostics Inc., Indianapolis, Ind.) to screen individuals for prostate cancer and to monitor individuals undergoing treatment for prostate cancer. Prostate cancer can be staged, for example, using a Partin Table and/or a Partin II Table (see Partin et al., 1994, Urology, 43:649-59 and http://www.theraseed.com/gloss.html for more information).

For the purpose of this invention, POH can be administered orally, transdermally, intravenously, intraperitoneally, or by implantation. The route of administration typically depends on a variety of factors, such as treatment environment and therapeutic goals. Administration of POH can be on a continuous or an intermittent basis. In addition, preparations for administration of POH can be suitably formulated to give controlled release of the compound. Preparations for intravenous and intraperitoneal administration can include sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, as well as alcohol, saline, and buffered solutions. Other additives such as, for example, antimicrobials, anti-oxidants, chelating agents, inert gases, steroids, anti-inflammatory agents, immunosuppressants, vasodilators, vasoconstrictors, and the like may also be present.

Tablets or capsules for oral administration can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Tablets can be coated by methods known in the art. Liquid preparations for oral administration can take the form of, for example, solutions, syrups or suspension, or they can be presented as a dry product for constitution with saline or other suitable liquid vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl- or propyl-p-hydroxybenzoates or sorbic acid). The preparations can also contain buffer salts, flavoring, coloring and sweetening agents as appropriate.

Preparations for transdermal administration are known in the art. Such transdermal preparations can be in the form of a scrotum patch or a patch for application on the back, abdomen, thighs or buttocks. A transdermal patch typically includes a soft flexible backing (e.g., polyester or polyester/ethylene-vinyl acetate copolymer), a reservoir (in some cases, the compound or composition, e.g., POH, can be deposited as a film on the ethylene-vinyl acetate copolymer or can be combined with, for example, alcohol and a gelling agent such as hydroxypropyl cellulose), and an adhesive backing made out of, for example, polyisobutylene and colloidal silicon dioxide (usually with a removable liner (e.g., silicone-coated polyester, or fluorocarbon diacrylate) to protect the adhesive until the patch is applied). A transdermal patch also can contain a formulation (e.g., polyisobutylene adhesive) to control the rate of release of the compound or composition.

Implantable devices are known in the art and can be in the form of a pellet or a seed containing or coated with a compound or composition, e.g., POH. A pellet or seed can be a metal alloy (e.g., cobalt, or palladium) or an inert plastic or other substance. A device for implantation in or near the prostate can be delivered using a delivery catheter (similar to brachytherapy) and can be deposited in or near the prostate transperineally, transrectally, or transurethrally. A transrectal ultrasound can be used in conjunction with implantation to visualize and image the prostate and the positioning of the implantable device.

According to the invention, an effective dose of POH is an amount that inhibits the transactivating ability of the androgen receptor, thereby inhibiting the proliferation of prostate cancer cells. Inhibition of the transactivating ability of the androgen receptor and the subsequent inhibition of the proliferation of prostate cancer cells can be determined using methods and assays described herein. It is anticipated that an effective dose of POH is from about 100 mg of POH per kg weight of the individual (mg/kg) to about 300 mg/kg. Toxicity and therapeutic efficacy of different doses of POH can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio of LD₅₀/ED₅₀. Doses of POH that exhibit high therapeutic indeces are preferred. An effective dose of POH can be delivered in a single dose or as multiple doses over a period of time.

The transactivating ability of the androgen receptor can be examined by evaluating the expression of genes whose transcription is regulated by androgen receptor binding. Such genes include PSA, h2k, NKX3.1, and ODC. The amount of transcript and/or protein of such genes in the presence and absence of the compound can be readily determined using art-routine methods such as those described herein. Alternatively, prostate cancer cells in culture can be made transgenic for one or more androgen-regulated genes and the expression of such transgenes can be evaluated in the presence and absence of a compound.

In addition, the invention provides methods of reducing the risk of recurrence of prostate cancer in an individual that previously had undergone treatment for prostate cancer. Such methods include administering an effective dose of POH to the individual such that the transactivating ability of the androgen receptor is inhibited. Inhibiting the transactivating ability of the androgen receptor inhibits the proliferation, and therefore the recurrence, of prostate cancer cells. Treatments for prostate cancer that an individual might undergo include hormone therapy, chemotherapy, radiation therapy and, oftentimes, a prostatectomy, in which part of all of the prostate gland is removed. A radical prostatectomy includes removal of the entire prostate as well as the seminal vesicles. Due to a high incidence of prostate cancer recurring, even following such treatments (including a radical prostatectomy), methods of the invention provide for administration of POH during or following such treatments. Administration of POH may be particularly useful following a radical prostatectomy.

The invention additionally provides for a method of treating an individual with benign prostatic hyperplasia (BPH). Individuals with BPH may present with prostatitis and/or difficulty urinating, and an enlarged prostate due to BPH is typically palpable during a digital rectal exam. Methods of the invention include identifying an individual with BPH, and administering a dose of POH or a derivative thereof to said individual effective to inhibit the transactivating ability of an androgen receptor. Such an inhibition of the androgen receptor's transactivating ability reduces the androgen receptor-mediated growth response and thereby treats the individual with BPH.

Methods of Screening Compounds

The invention provides for methods of screening for compounds that inhibit the proliferation of prostate cancer cells by decreasing the transactivating ability of the androgen receptor. Screening methods are one of the fundamental tools used in molecular biology for rapid and efficient evaluation of compounds. Screening methods of the invention include contacting prostate cancer cells with a compound under conditions and for a time sufficient to allow the compound to enter the cell, and determining the transactivating ability of the androgen receptor. Generally, decreased transactivating ability of the androgen receptor in cells compared to cells not contacted with the compound indicates a compound that inhibits the proliferation of prostate cancer cells. Such compounds can be evaluated using prostate cancer cells in culture, such as LNCaP or LAPC-4 cells, or can be evaluated using a cell-free system.

Methods of determining the transactivating ability of the androgen receptor are described above. Expression of a gene encoding an androgen receptor in prostate cancer cells can be examined in the presence and absence of a compound using Northern blot analysis (to evaluate transcription) and/or Western blot analysis (to evaluate translation). Techniques to isolate RNAs and proteins from cells as well as methods of separation (e.g., electrophoretically) are well known and routine in the art. Androgen receptor mRNA can be detected by hybridization with a labeled oligonucleotide probe that is corplementry to a portion of the androgen receptor transcript. Androgen receptor proteins can be detected by contacting proteins from a cell with a labeled agent that selectively binds to the androgen receptor protein. Conditions for allowing and detecting hybridization of nucleic acids or binding of antibodies to proteins are well known in the art. Antibodies that have binding affinity to androgen receptor proteins are commercially available (e.g., from Research Diagnostics Inc. (Flanders, N.J.) and Alpha Diagnostic International (San Antonio, Tex.)). The term “label”, with regard to an oligonucleotide probe or an antibody is intended to encompass direct labeling of the oligonucleotide or antibody by coupling a detectable substance to the oligonucleotide or antibody, as well as indirect labeling of the oligonucleotide or antibody by reactivity with a detectable substance. Examples of labels and detectable substances are well known in the art. Additional methods to detect androgen receptor mRNA (e.g., RT-PCR or dot blots) or protein (e.g., immunoassays or chromatography) are well known and also practiced routinely in the art.

The ability of the androgen receptor to translocate to the nucleus also can be evaluated in the presence and absence of a compound to determine if the compound inhibits the nuclear localization of the androgen receptor. Nuclei are typically isolated using an appropriate gradient such as a sucrose gradient, a percol gradient, or the like. The nuclei can be lysed (for example, by exposure to sonication, or ultrasound waves) and androgen receptor protein can be detected using routine methods such as Western blotting. Nuclear translocation also can be examined using, for example, immunocytochemistry to identify androgen receptor protein in the nucleus and/or outside of the nucleus.

In addition, the amount of c-jun protein can be evaluated as an indicator of androgen receptor activity. When overexpressed, c-jun has been shown to inhibit the transactivating ability of the androgen receptor. c-jun is a partner with c-fos in the transcription factor AP-1. Increased evidence suggests that the function of the androgen receptor may be affected by an interaction with AP-1.

Compositions and Articles of Manufacture

The invention provides compositions that include POH or a derivative thereof and at least one other compound selected for its particular mechanism of action on the androgen receptor. The mechanism of action exerted by the other compound(s) can be one or more of the following: inhibition of the expression of a gene encoding an androgen receptor; inhibition of the nuclear localization of an androgen receptor; or inhibition of the transactivating ability of an androgen receptor. Representative compounds exhibiting such mechanisms of action include the following: resveratrol, and omega-3 fatty acids (transactivating ability); silymarin (nuclear localization); flufenamic acid, tea polyphenols (e.g., (−)-epigallocatechin gallate (EGCG)), and quercetin (expression); and numerous anti-androgen compounds (e.g., bicalutamide, flutamide, nilutamide, or cyproterone).

Compositions containing POH can be formulated for delivery to the prostate. In one aspect, POH is formulated for transdermal delivery to the prostate. In another aspect, compositions containing POH can be formulated for implantation in or near the prostate. Delivery of compositions containing POH directly to the prostate of an individual inhibits the transactivating ability of the androgen receptor. Formulations for administration of POH described above and apply as well to the disclosed compositions containing POH.

A composition containing POH can be in any form provided the composition can be administered to an individual in an amount and for a duration effective to inhibit the transactivating ability of the androgen receptor gene, thereby inhibiting the proliferation of prostate cancer cells. Pharmaceutically acceptable carriers include solvents, dispersion media, coatings, antibacterial and anti-fungal agents, isotonic and absorption delaying agents and the like, appropriate to specific routes of administration.

POH compositions of the invention that are effective for inhibiting transactivating ability of the androgen receptor as described herein can be combined with packaging material and sold as a kit (i.e., an article of manufacture). Components and methods for producing articles of manufactures are well known. In addition to a composition containing articles of manufacture can include oligonucleotide probes, antibodies, and/or other useful agents for determining the transactivating ability of the androgen receptor. Instructions describing how the composition can be used for inhibiting the transactivating ability of the androgen receptor to thereby inhibit the proliferation of prostate cancer cells can be included in such kits.

In accordance with the present invention, there may be employed conventional molecular biology, microbiology, biochemical and recombinant DNA techniques within the skill of the art. Such techniques are explained fully in the literature. The invention will be further described in the following examples, which do not limit the scope of the invention described in the claims.

### EXAMPLES

### Example 1

### Cell Cultures Cell Proliferation Assays and PSA and hK2 Quantification Assays

A human prostate cancer cell line, LNCaP (American Type Culture Collection (ATCC), Manassas, Va.), was grown in RPMI 1640 medium (Mediatech, Herndon, Va.) supplemented with 5% fetal bovine serum (FBS) and 5% CO₂ at 37° C. until reaching approximately 50-70% confluence. The media were changed to serum-free RPMI 1640 at 24 hrs prior to performing experiments to deplete undesired steroids. Cells were then treated with 5% charcoal-stripped FBS RPMI 1640 containing POH (all from Sigma (St. Louis, Mo.), dissolved in DMSO) at indicated concentrations with or without 1 nM of mibolerone (Mib) (from New England Nuclear (St. Louis, Mo.), dissolved in ethanol), a non-metabolizable synthetic androgen. Equivalent amounts of solvent were added to control cells.

LNCaP cells were seeded at 4×10⁴/well in 24-well dishes and treated with POH at indicated concentrations in the presence of 1 nM Mib. Five days later, cell proliferation was measured using an MTS assay kit (Promega, Madison, Wis.), and PSA and hK2 levels in spent media were determined by the Tandem-E PSA kit (Hybritech Inc., San Diego, Calif.) or Mayo's hK2 assay (Zhang et al., 1999, Endocrin,140:1665-71). Protein levels of PSA and hK2 were normalized to the MTS measurements.

### Example 2

### Western Blot Analysis

LNCaP cells were treated with the indicated concentrations of POH in the presence of 1 nM Mib for 24 hrs. Cells were then harvested, and whole-cell lysates and nuclear extracts were prepared as described (Mitchell et al., 1999, Cancer Res., 59:5892-5). Western blot analysis was performed according to the protocol described (Id). A mouse antibody against the human androgen receptor (1:1000 or 1:2000 dilution) (Pharmingen, San Diego, Calif.) or human tubulin (1:10,000 dilution) (Santa Cruz, Santa Cruz, Calif.) was used as the primary antibody. Ponceau S staining was used for monitoring protein loading and transfer efficiency (Id).

### Example 3

### DNA Constructs

The 6 Kb PSA promoter and the androgen receptor promoter constructs (pGL3 SV40, pGL3 SV40-3 ARE, pGL3 or PSA promoter/pGL3) were described previously (Id). To make an hk2 androgen responsive element (hk2 ARE) construct, a DNA fragment containing three copies of hk2 ARE (5′-GGAACATATTGTATT-3′ (SEQ ID NO:1)) was synthesized by the Mayo Molecular Core Facility. The synthesized fragment, including SacI and XhoI restriction enzyme sites at the 5′ and 3′-end, respectively, was digested with SacI and XhoI according to manufacturer's instructions and inserted into a pre-cut pGL3-Promoter vector (Promega). The fidelity of this construct was confirmed by DNA sequencing.

### Example 4

### Transient Transfection Assays

LNCaP cells in duplicate plates were co-transfected with a CMV-β galactosidase (β-gal) expression vector (0.3 μg/plate) and one of the following: a pGL3-Basic luciferase vector (Promega) containing the PSA promoter (nucleotides 1-5836 of GenBank Accession No. U37672), a pGL3-Promoter luciferase vector (Promega) containing three copies of hk2 ARE, a PGL3 vector or PSA promoter/PGL3 vector. Transfections were performed using a liposome method with dimethyldioctadecyl-ammonium bromide (Sigma) and L-lecithin (Sigma) (4:10). Cells were then treated with POH in the presence or absence of 1 nM Mib for 24 hrs. Cell extracts were prepared and used for luciferase and β-gal assays (Promega). The β-gal activity was used as a control for transfection efficiency and for normalization of luciferase activity. The above experiments were repeated three times.

### Example 5

### Effect of POH on the Androgen Receptor

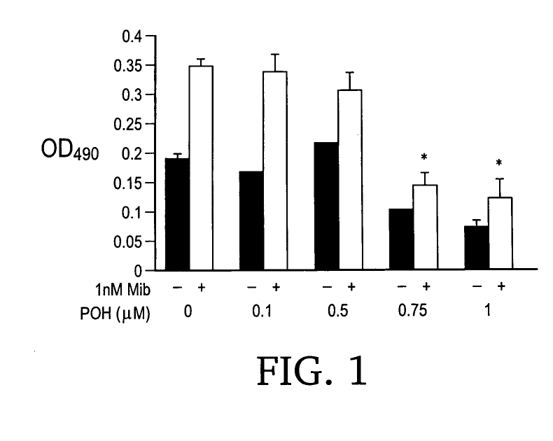

The presence of Mib significantly increased LNCaP cell proliferation while POH significantly inhibited the proliferation of LNCaP cells (FIG. 1). Results were analyzed by 2-tailed Student's t-test. A p<0.05 was accepted as the level of significance.

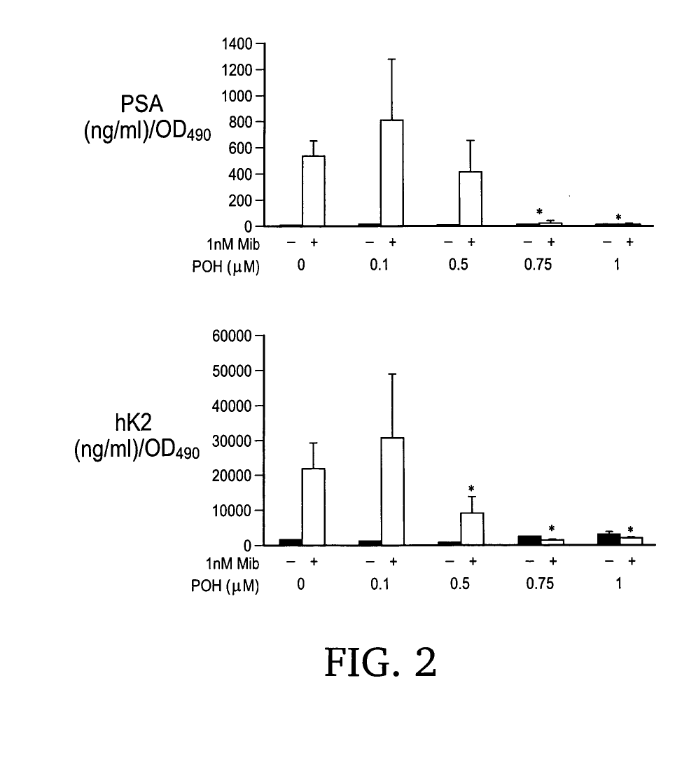

Androgen up-regulated proteins prostate-specific antigen (PSA) and hK2, were used as monitors of the androgen receptor activity. Their promoters contain androgen-responsive elements (AREs) for androgen receptor binding. As shown in FIGS. 2A and 2B, POH inhibited the accumulation of PSA and hK2 protein in LNCaP cells stimulated by androgens.

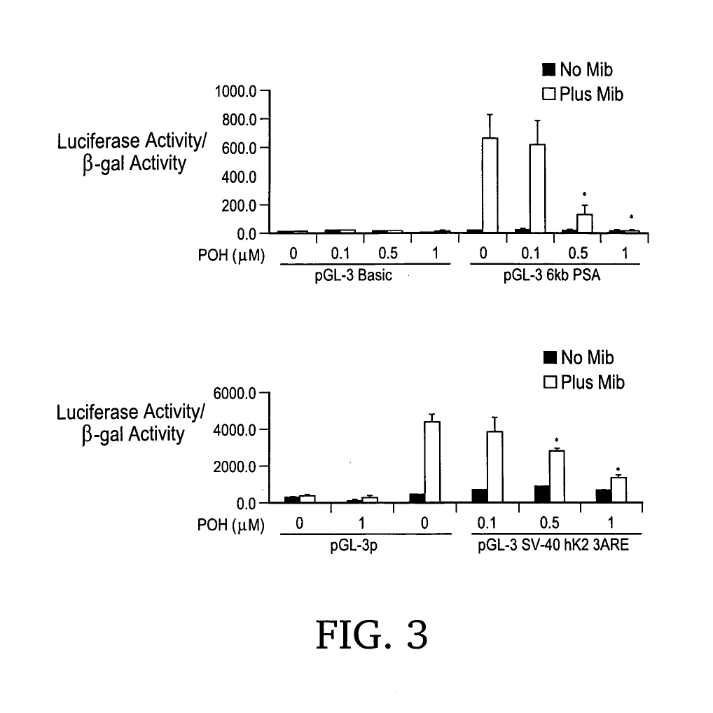

Since the androgen receptor is the major regulator of PSA expression, a luciferase reporter gene containing the PSA promoter or containing a minimum SV40 promoter and 3 copies of hK2 ARE were transfected into LNCaP cells. POH significantly reduced the androgenic inducibility of the PSA promoter (FIG. 3A), demonstrating that androgen receptor function is impaired by POH. As shown in FIG. 3B, POH inhibited the ARE-regulated luciferase activity. These results demonstrate that POH inhibits androgen receptor-mediated transcriptional activation.

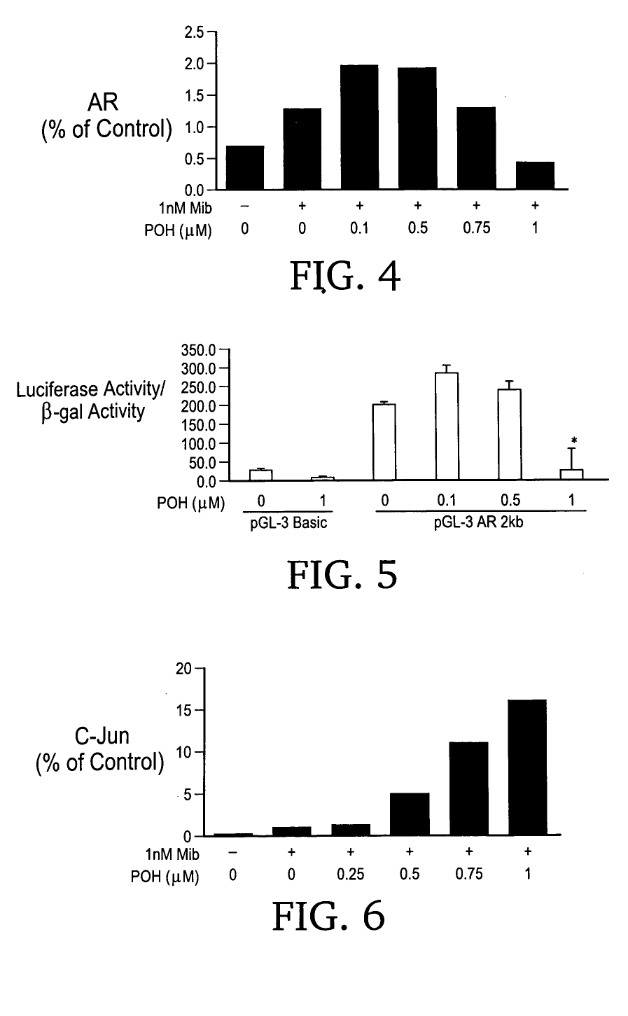

Expression of the gene encoding the androgen receptor in the presence of POH was examined (FIGS. 4 & 5). The androgen-enhanced androgen receptor protein levels were not affected by POH until POH reached a concentration of 1 μM. Moreover, a luciferase reporter plasmid containing the androgen receptor promoter was transfected into LNCAP cells, and androgen receptor promoter activity also was not affected by POH below 1 μM. POH at 1 μM drastically reduced androgen receptor promoter activity. These results seem to suggest that POH below 1 μM can impair the androgen receptor's function without affecting its expression levels, whereas POH at 1 μM represses the androgen receptor's function by inhibiting its expression.

In order to ascertain how androgen receptor function was being inhibited by POH at levels below 1 μM, c-jun was examined. FIG. 6 shows that the level of c-jun increased with POH treatment up to 3.5 times the control levels. The graph depicts the normalized data. This experiment was repeated twice and representative data is shown. Previous studies (Murtha et al., 1997, Prostate, 33:264-70; Lobaccaro et al., 1999, Endocrin, 140:350-7) showed that stimulated overexpression of c-jun protein can inhibit the function of the androgen receptor, because c-jun binds the androgen receptor and competes with other co-activators necessary for the androgen receptor's transactivating ability. Results from experiments herein suggest that POH induces the overexpression of c-jun that then represses the transactivating ability of the androgen receptor.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-19. (Canceled)

20. A method of treating an individual with prostate cancer or at risk of developing prostate cancer, comprising the steps of:
identifying an individual with prostate cancer or at risk of developing prostate cancer;
administering a dose of POH or a derivative thereof to said individual effective to inhibit the proliferation of prostate cancer cells; and
monitoring said individual for a dose-dependent reduction in prostate-specific antigen (PSA) levels, wherein said dose-dependent reduction in PSA correlates with a dose-dependent inhibition of said transactivating ability of said androgen receptor.

21. The method of claim 20, further comprising:
monitoring human glandular kallikrein (hK2) levels in said individual, wherein a reduction in hK2 correlates with an inhibition of said transactivating ability of said androgen receptor.

22. The method of claim 20, further comprising:
adjusting, if necessary, said dose of POH or a derivative thereof to achieve or maintain said dose-dependent reduction in PSA.

23. A method of treating an individual with prostate cancer or at risk of developing prostate cancer, comprising the steps of:
identifying an individual with prostate cancer or at risk of developing prostate cancer;
administering a dose of POH or a derivative thereof to said individual effective to inhibit the proliferation of prostate cancer cells; and
monitoring human glandular kallikrein (hK2) levels in said individual, wherein a reduction in hK2 correlates with an inhibition of said transactivating ability of said androgen receptor.

24. The method of claim 23, further comprising:
monitoring said individual for a dose-dependent reduction in prostate-specific antigen (PSA) levels, wherein said dose-dependent reduction in PSA correlates with a dose-dependent inhibition of said transactivating ability of said androgen receptor.

25. The method of claim 23, further comprising:
adjusting, if necessary, said dose of POH or a derivative thereof to achieve or maintain said reduction in hK2.

26. A method of reducing the risk of recurrence of prostate cancer in an individual, wherein said individual previously had been treated for prostate cancer, comprising the step of:
administering a dose of POH or a derivative thereof to said individual effective to inhibit the proliferation of prostate cancer cells; and
monitoring said individual for a dose-dependent reduction in prostate-specific antigen (PSA) levels, wherein said dose-dependent reduction in PSA correlates with a dose-dependent inhibition of said transactivating ability of said androgen receptor.

27. The method of claim 26, further comprising
monitoring human glandular kallikrein (hK2) levels in said individual, wherein a reduction in hK2 correlates with an inhibition of said transactivating ability of said androgen receptor.

28. The method of claim 26, further comprising:
adjusting, if necessary, said dose of POH or a derivative thereof to achieve or maintain said dose-dependent reduction in PSA.

29. A method of reducing the risk of recurrence of prostate cancer in an individual, wherein said individual previously had been treated for prostate cancer, comprising the step of:
administering a dose of POH or a derivative thereof to said individual effective to inhibit the proliferation of prostate cancer cells; and
monitoring human glandular kallikrein (hK2) levels in said individual, wherein a reduction in hK2 correlates with an inhibition of said transactivating ability of said androgen receptor.

30. The method of claim 29, further comprising:
monitoring said individual for a dose-dependent reduction in prostate-specific antigen (PSA) levels, wherein said dose-dependent reduction in PSA correlates with a dose-dependent inhibition of said transactivating ability of said androgen receptor.

31. The method of claim 29, further comprising:
adjusting, if necessary, said dose of POH or a derivative thereof to achieve or maintain said reduction in hK2.

32. A method of treating an individual with benign prostatic hyperplasia (BPH) or at risk of developing BPH, comprising the steps of:
identifying an individual with BPH or at risk of developing BPH;
administering a dose of POH or a derivative thereof to said individual effective to inhibit the proliferation of BPH cells; and
monitoring said individual for a dose-dependent reduction in prostate-specific antigen (PSA) levels, wherein said dose-dependent reduction in PSA correlates with a dose-dependent inhibition of said transactivating ability of said androgen receptor.

33. The method of claim 32, further comprising:
monitoring human glandular kallikrein (hK2) levels in said individual, wherein a reduction in hK2 correlates with an inhibition of said transactivating ability of said androgen receptor.

34. The method of claim 32, further comprising:
adjusting, if necessary, said dose of POH or a derivative thereof to achieve or maintain said dose-dependent reduction in PSA.

35. A method of treating an individual with benign prostatic hyperplasia (BPH) or at risk of developing BPH, comprising the steps of:
identifying an individual with BPH or at risk of developing BPH; and
administering a dose of POH or a derivative thereof to said individual effective to inhibit the proliferation of BPH cells; and
monitoring human glandular kallikrein (hK2) levels in said individual, wherein a reduction in hK2 correlates with an inhibition of said transactivating ability of said androgen receptor.

36. The method of claim 35, further comprising:
monitoring said individual for a dose-dependent reduction in prostate-specific antigen (PSA) levels, wherein said dose-dependent reduction in PSA correlates with a dose-dependent inhibition of said transactivating ability of said androgen receptor.

37. The method of claim 35, further comprising:
adjusting, if necessary, said dose of POH or a derivative thereof to achieve or maintain said reduction in hK2.
